(19) United States
(12) Patent Application Publication
(10) Pub. No.: US 2007/0118119 A1
(43) Pub. Date: May 24, 2007

(54) Methods and device for dynamic stabilization

(75) Inventor: Hugh D. Hestad, Edina, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/283,197

(22) Filed: Nov. 18, 2005

(51) Int. Cl. A61F 2/30

(52) U.S. Cl. 606/61

(57) ABSTRACT

A method of stabilizing a motion segment of the spine. A posterior incision is formed lateral to a spinous process of the spine. A length and width of the incision is stretched. A portal having a sleeve portion defining an open inner area is inserted into the incision. The pedicles are exposed through the inner area of the portal. Bone-engaging members are attached to adjacent pedicles with a gap therebetween. A cord is attached to a first of the bone-engaging members. A spacer is advanced over the cord into the gap between the bone-engaging members. The cord is tensioned and attached to the other of the bone-engaging members.

### TECHNICAL FIELD

This disclosure relates generally to methods and devices for accessing an area of a patient's spinal column during a surgical procedure. More particularly, this disclosure relates to an instrument that provides an access opening to the spinal column.

### BACKGROUND

A wide variety of surgical techniques have been used to access the spinal column in spinal surgery procedures. For example, some techniques included making an incision in the patient's back and distracting or separating tissue and muscle to expose a wide area of the spine in order to perform the spinal surgery procedure. Such techniques often result in excessive invasions into the patient's spine and back region causing major damage to the normal anatomy, and significant and dangerous blood loss.

In an attempt to minimize risks associated with spinal surgery procedures, some surgical techniques have been developed wherein only portions of the spinal column area are accessed during various stages of the surgical procedure. In these procedures, a smaller incision can be used to access the portion of the spinal column area. However, access to only a portion of the spinal column area does not provide sufficient access for all surgical procedures.

In general, improvement has been sought with respect to such surgical techniques, generally to better provide sufficient accessibility to a spinal column area while minimizing anatomical trauma and blood loss.

### SUMMARY

In one embodiment, the present invention is a method of stabilizing a motion segment of the spine. A posterior incision is formed lateral to a spinous process of the spine. A length and width of the incision is stretched. A portal having an open inner area sized and shaped to provide access to the pedicles of adjacent vertebrae and having a long axis aligned with an axis extending between the pedicles is inserted into the incision. Finally, a dynamic spinal stabilization device is implanted onto the pedicles through the open inner area of the portal.

In another embodiment, the present invention is a method of stabilizing a motion segment of the spine. A posterior incision is formed lateral to a spinous process of the spine. Incrementally larger sleeve members are inserted into the incision to stretch a length and a width of the incision. A portal having an open inner area sized and shaped to provide access to the pedicles of adjacent vertebrae and having a long axis aligned with an axis extending between the pedicles is inserted into the incision. A dynamic spinal stabilization device is implanted onto the pedicles through the open inner area of the portal.

In yet another embodiment, the present invention is a method of stabilizing a motion segment of the spine. A posterior incision is formed lateral to a spinous process of the spine. A portal having a sleeve portion defining an open inner area is inserted into the incision. Finally, a dynamic stabilization device is implanted onto the spine through the open inner area of the portal. This is done by attaching a pair of bone-engaging members to adjacent pedicles with a gap therebetween, attaching a flexible cord to a first of the bone-engaging members, advancing a spacer over the cord into the gap between the bone-engaging members, tensioning the cord and attaching the cord to the other of the bone-engaging members.

A variety of examples of desirable product features or methods are set forth in part in the description that follows, and in part will be apparent from the description, or may be learned by practicing various aspects of the disclosure. The aspects of the disclosure may relate to individual features as well as combinations of features. It is to be understood that both the foregoing general description and the following detailed description are explanatory only, and are not restrictive of the claimed invention.

### BRIEF DESCRIPTION OF THE DRAWINGS

FIG. 1 is a perspective view of one embodiment of a surgical instrument according to the principals of the present disclosure, shown in a nested configuration.

FIG. 2 is a perspective view of the surgical instrument of FIG. 1, shown partially exploded.

FIG. 3 is a perspective view of the components of the surgical instrument of FIG. 2, shown disassembled.

FIG. 4 is a top plan view of one embodiment of a blade member according to the principals of the present disclosure, and shown in FIG. 3.

FIG. 5 is a front elevational view of the blade member of FIG. 4.

FIG. 6 is a side elevational view of the blade member of FIG. 4.

FIG. 7 is a top plan view of one embodiment of an inner portal member according to the principals of the present disclosure, and shown in FIG. 3.

FIG. 8 is a front elevational view of the inner portal member of FIG. 7.

FIG. 9 is a side elevational view of the inner portal member of FIG. 7.

FIG. 10 is top plan view of one embodiment of an intermediate portal member according to the principals of the present disclosure, and shown in FIG. 3.

FIG. 11 is a front elevational view of the intermediate portal member of FIG. 10.

FIG. 12 is a side elevational view of the intermediate portal member of FIG. 10.

FIG. 13 is a rear elevational view of one embodiment of an outer portal member according to the principals of the present disclosure, and shown in FIG. 3.

FIG. 14 is a cross-sectional view of the outer portal member of FIG. 13, taken along line 14-14.

FIG. 15 is a cross-sectional view of the outer portal member of FIG. 14, taken along line 15-15.

FIG. 16 is a top plan view of another embodiment of an outer portal member according to the principals of the present disclosure, shown in a retracted position.

FIG. 17 is a top plan view of the outer portal member of FIG. 16, shown in a distended position.

FIG. 18 is a perspective view of the outer portal member of FIG. 17.

FIG. 19 is a side elevational view two vertebrae.

FIG. 20 is a top plan view of one of the two vertebrae of FIG. 19.

FIG. 21 is a side elevation view of the outer portal member according to another embodiment of the present invention.

FIG. 22 is a top plan view of the outer portal member of FIG. 21 relative to one of the two vertebrae of FIG. 19.

FIG. 23 is a side elevation view of the outer portal member according to another embodiment of the present invention.

FIG. 24 is a top plan view of the outer portal member of FIG. 23 relative to one of the two vertebrae of FIG. 19.

FIG. 25 is a side view of a dynamic spinal stabilization device for use with various embodiments of the spinal access instrument of FIGS. 1-18 and 21-24, shown disassembled.

FIG. 26 is a posterior view of a patient showing the outer portal member installed to access adjacent vertebrae and showing a spacer template.

FIG. 27 is a side partial cross-sectional view of the installed outer portal member of FIG. 26 taken along line A-A showing the bone-engaging members installed onto the pedicles and a measuring device for measuring the space between adjacent bone-engaging members.

FIG. 28 is a side partial cross-sectional view of the installed outer portal member of FIG. 27 showing the cord of FIG. 25 threaded through a first of the bone-engaging members.

FIG. 29 is a side partial cross-sectional view of the installed outer portal member of FIG. 28 showing the spacer of FIG. 25 threaded onto the cord.

FIG. 30 is a side partial cross-sectional view of the installed outer portal member of FIG. 29 showing the cord threaded through the second of the bone-engaging members and a depressor employed to position the spacer between the bone-engaging members.

FIG. 31 is a posterior view of a patient showing a laterally offset incision adjacent the incision of FIG. 26.

### DETAILED DESCRIPTION

Reference will now be made in detail to various features of the present disclosure that are illustrated in the accompanying drawings. Wherever possible, the same reference numbers will be used throughout the drawings to refer to the same or like parts.

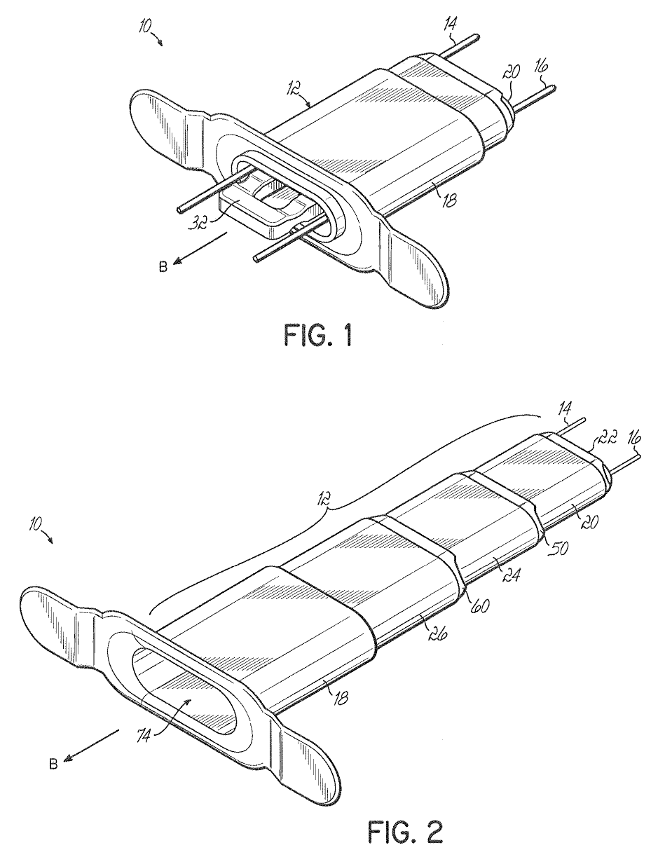

FIGS. 1-18 illustrate surgical instrument embodiments having features that are examples of how inventive aspects in accordance with the principals of the present disclosure may be practiced. Preferred features of the embodiments are adapted for providing a sufficient access opening to a spinal column area while minimizing risks associated with spinal surgery, such as incisional invasiveness, trauma, and blood loss.

Referring to FIG. 1, one embodiment of the spinal access instrument 10 is illustrated in complete assembly. The spinal access instrument is used to dissect skin tissue and muscle and provide a sufficiently sized opening for accessing a patient's spinal column. A sufficiently sized opening is an opening that is large enough to perform the desired surgical procedure. Preferably the opening provides access to a spinal column area or region such that the surgical procedure can be performed without having to provide more than one incision or opening.

For example, when performing a spinal procedure involving placement of pedicle screws (schematically represented in FIG. 20 by dashed lines), preferably the accessed spinal column area or region includes first and second pedicle sites. As shown in FIGS. 19 and 20, the first and second pedicle sites or locations are the two sites (A1, A2 (hidden) or B1, B2) that are vertically aligned on upper and lower vertebral bodies V1, V2. That is, the access opening is preferably sized to provide access to the spinal column area including both the first pedicle site (e.g. B1) and the second pedicle site (e.g. B2) of two adjacent vertebrae.

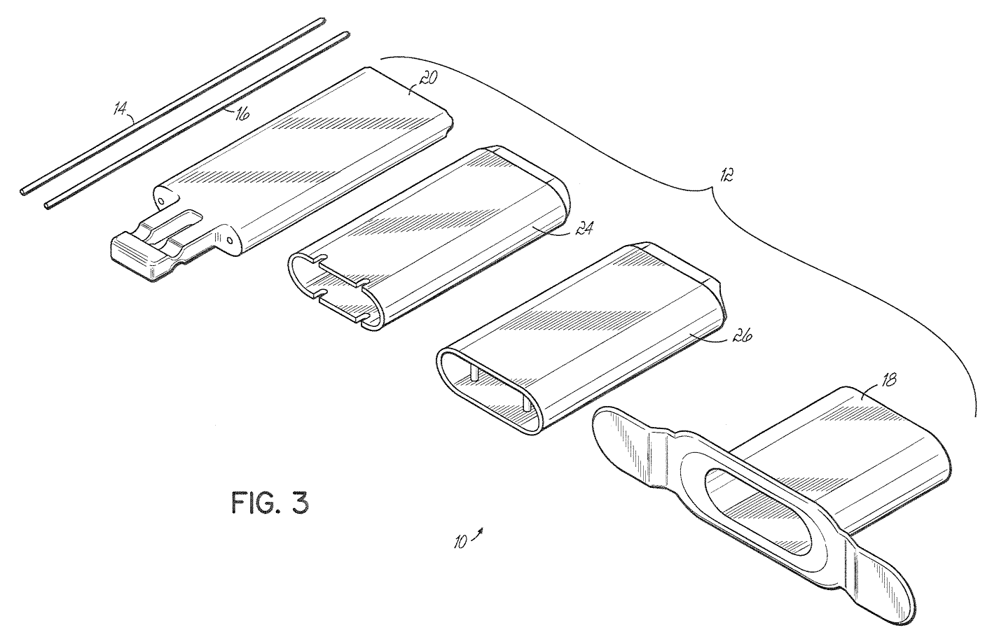

Referring back to FIG. 1, the surgical instrument 10 generally includes a nested arrangement 12, a first guide or placement wire 14, and a second guide or placement wire 16. As shown in FIGS. 2 and 3, the nested arrangement 12 of the spinal access instrument 10 includes a plurality of components or members sized so that each member fits with the other members in a nested configuration (as shown in FIG. 1). In the nested configuration, each of the members at least partially contains or is at least partially contained within the other members. The plurality of nested members includes at least one portal member (18, 24 or 26) and a dissector or blade member 20. As will be discussed in greater detail, the blade member 20 is used to provide an initial incision and the portal member provides access to the spinal column area through the incision.

Preferably the nested arrangement 12 is configured to incrementally provide an access opening to the spinal column area. What is meant by “incrementally provide an access opening” is that the arrangement provides an initial opening, and thereafter can be used to expand the opening (i.e. increase the cross-sectional area of the opening) as needed. By incrementally expanding the opening, surgical trauma and blood loss is minimized. In contrast, some existing procedures involve making an incision much wider than the incision needed by the present disclosure. The wider incision is needed in some existing procedures so that the skin tissue and muscle can be separated or pulled apart to adequately expose the spinal column area. This excessive invasion often results in anatomical trauma to the tissue or muscle and high blood loss.

In the illustrated embodiment of FIGS. 2 and 3, the nested arrangement 12 includes the blade member 20, and second, third, and fourth sleeve members 24, 26, and 18; although any number of sleeve members can be used in accord with the present disclosure. The second sleeve member or inner portal member 24 is slidably positionable over the blade member 20. The second sleeve member 24 is sized to expand the area of initial incision created by the blade member 20 to a second opening area. The second opening area is generally defined by the outer perimeter of the second sleeve member 24. The third sleeve member or intermediate portal member 26 is slidably positionable over the second sleeve member 24. The third sleeve member 26 is sized and configured to expand the access opening from the second opening area defined by the second sleeve member 24 to a third opening area. The third opening area is generally defined by the outer perimeter of the third sleeve member 26. Finally, the fourth sleeve member or outer portal member 18 is slidably positionable over the third sleeve member 26. The outer portal member 26 is sized and configured to expand the access opening from the third opening area defined by the third sleeve member 26 to a final opening area. The final opening area is generally defined by the outer perimeter of the outer portal member 18.

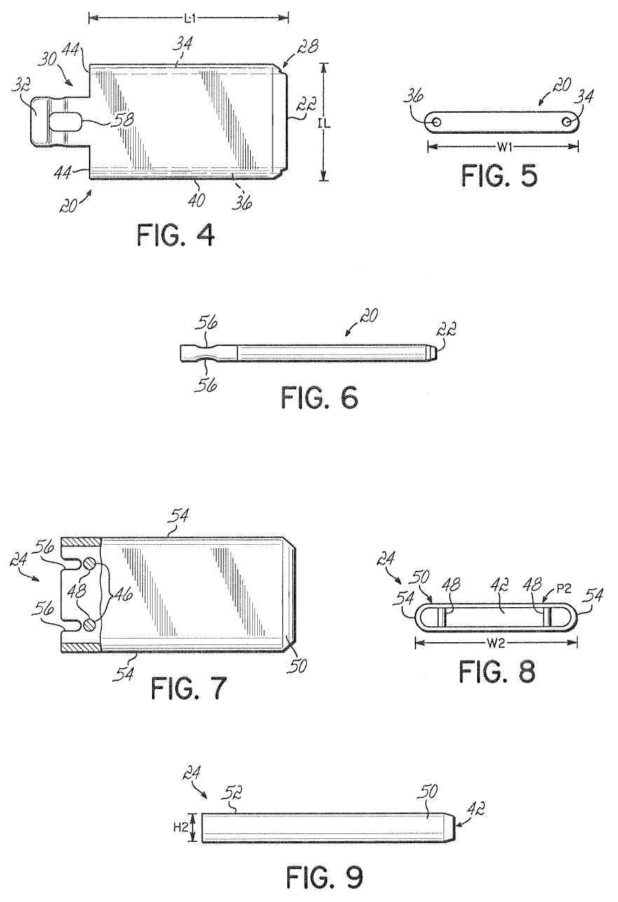

Referring now to FIGS. 4-6, the blade member 20 of the surgical instrument 10 includes a first end 28 and a second end 30. The first end 28 of the blade member 20 is typically a solid construction defining a blade edge 22. The blade edge 22 is configured to provide an initial incision of length IL (FIG. 4) in the skin tissue and muscle of a patient. A handle 32 is located at the second end 30 opposite the first end 28 of the blade member 20. As shown in FIGS. 4 and 6, the handle includes recessed areas 56 and an aperture 58 for gripping. The handle 32 can include a variety of shapes and geometries configured for gripping and moving the blade member 20 during use.

In general, the blade member 20 has an overall width W1, an overall height H1, and an overall length L1, although the disclosed principles can be applied in a variety of sizes and applications. The width W1 of the blade member 20 is shown in FIG. 5, and is preferably between 19 mm and 58 mm (0.75 inches and 2.25 inches); more preferably between 38 mm and 45 mm (1.5 inches and 1.75 inches). The height H1 of the blade member 20 is shown in FIG. 6, and is preferably between 4 mm and 10 mm (0.175 inches and 0.375 inches); more preferably between 5 mm and 7 mm (0.200 inches and 0.250 inches). The length L1 of the blade member 20 is generally defined between the first end 28 and the second end 30 of the blade member 20, excluding the handle 32. The length L1 of the blade member 20 is preferably between 88 mm and 140 mm (3.5 inches and 5.5 inches); more preferably between 101 mm and 127 mm (4.0 inches and 5.0 inches).

As shown in FIGS. 4 and 5, the blade member 20 includes first and second apertures 34, 36 extending along the length L1 of the blade member 20. The first and second aperture 34, 36 are offset from edges 38, 40 of the blade member 20 and extend from the first end 28 to the second end 30 of the blade member 20. Each of the first and second apertures 34, 36 is sized and configured for receipt of the corresponding first and second placement wires 14, 16 (FIG. 2). In the illustrated embodiment, the first and second placement wires 14, 16 are approximately 2 mm (0.08 inches) in diameter; correspondingly the first and second apertures 34, 36 are approximately 2.3 mm (0.09 inches) in diameter.

Referring now to FIGS. 7-9, the second sleeve member or inner portal member 24 of the nested arrangement 12 is illustrated. The second sleeve member 24 is generally a tubular construction having a first end 50 and a second end 52. The tubular construction of the second sleeve member defines an elongated aperture 42 sized and configured for receipt of the blade member 20. In particular, the second sleeve member 24 fits over the handle and slides along the blade member to nest with or cover the blade member 20. The first end 50 of the second sleeve member 24 is tapered. In use, the tapered first end 50 assists in gradually expanding the access opening from the initial area of the incision created by the blade member 20 to the second opening area defined by the outer perimeter P2 (FIG. 8) of the second sleeve member 24.

The second sleeve member 24 is configured to slide over the blade member 20 until shoulders 44 (FIG. 4) of the blade member 20 contact stop structures 46 of the second sleeve member 24. In the illustrated embodiment, the stop structures 46 include pins 48 positioned within the elongated aperture 42. The pins 48 are positioned adjacent to the second end 52 of the second sleeve member 24. Each of the pins 48 is offset from sidewalls 54 of the second sleeve member 24 so that when assembled as shown in FIGS. 1 and 2, the first and second placement wires 14, 16 extend between the pins 48 and the sidewalls 54 of the second sleeve member 24.

In general, the second sleeve member 24 has an overall width W2, an overall height H2, and an overall length L2, although the disclosed principles can be applied in a variety of sizes and applications. The width W2 of the second sleeve member 24 is shown in FIG. 8, and is preferably between 24 mm and 63 mm (0.95 inches and 2.45 inches); more preferably between 43 mm and 50 mm (1.70 inches and 1.95 inches). The height H2 of the second sleeve member 24 is shown in FIG. 9, and is preferably between 9 mm and 15 mm (0.375 inches and 0.575 inches); more preferably between 10 mm and 12 mm (0.400 inches and 0.450 inches). The length L2 of the second sleeve member 24 is generally defined between the first end 50 and the second end 52 of the second sleeve member 24. The length L2 of the second sleeve member is preferably between 95 mm and 146 mm (3.75 inches and 5.75 inches); more preferably between 107 mm and 134 mm (4.25 inches and 5.25 inches). The outer perimeter P2 of the second sleeve member 24 defines the second access opening area; the second access opening area is generally between 180 and 716 square mm (0.28 and 1.11 square inches).

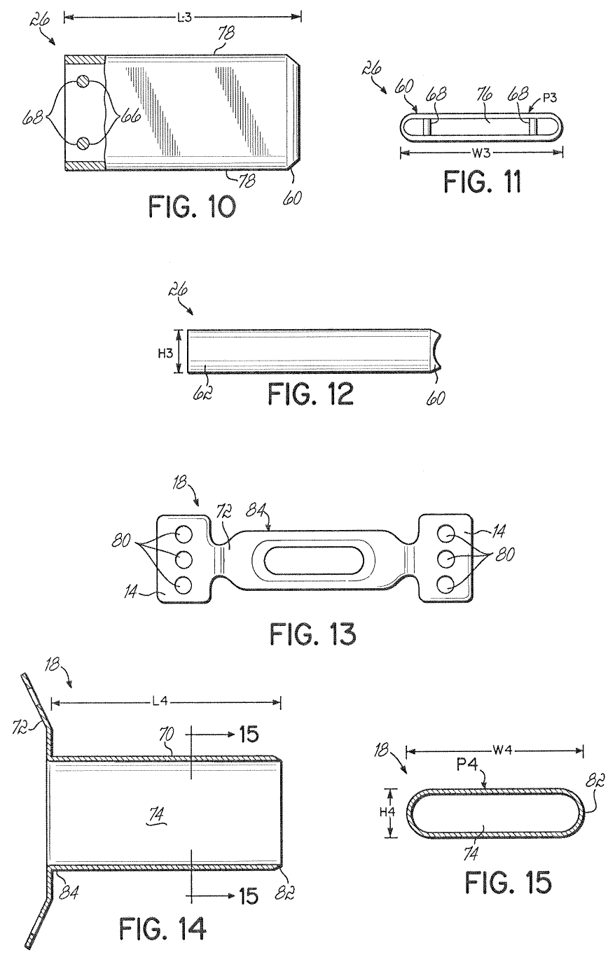

Referring now to FIGS. 10-12, the third sleeve member or intermediate portal member 26 of the nested arrangement 12 is illustrated. The third sleeve member 26 is also generally a tubular construction having a first end 60 and a second end 62. The tubular construction of the third sleeve member 26 defines an elongated aperture 76 sized and configured for receipt of the second sleeve member 24. In particular, the third sleeve member 26 fits over the second sleeve member 24 to nest with or cover the second sleeve member 24. Similar to the second sleeve member, the first end 60 of the third sleeve member is tapered to assist in gradually expanding the access opening from the second opening area to the third opening area defined by the outer perimeter P3 of the third sleeve member 26.

The third sleeve member 26 slides over the second sleeve member 24 until notches 56 (FIG. 7) of the second sleeve member 24 contact stop structures 66 of the third sleeve member 26. In the illustrated embodiment, the stop structures 66 include pins 68 positioned within the elongated aperture 76. The pins 68 are positioned adjacent to the second end 62 of the third sleeve member 26. Each of the pins 68 is offset from sidewalls 78 of the third sleeve member 26 so that when assembled as shown in FIG. 2, the first and second placement wires 14, 16 extend between the pins 68 and the sidewalls 78 of the third sleeve member 26.

In general, the third sleeve member 24 has an overall width W3, an overall height H3, and an overall length L3, although the disclosed principles can be applied in a variety of sizes and applications. The width W3 of the third sleeve member 26 is shown in FIG. 11, and is preferably between 27 and 66 mm (1.08 inches and 2.58 inches); more preferably between 46 mm and 53 mm (1.83 inches and 2.08 inches). The height H3 of the third sleeve member 26 is shown in FIG. 12, and is preferably between 17 mm and 23 mm (0.675 inches and 0.875 inches); more preferably between 17 mm and 19 mm (0.700 inches and 0.750 inches). The length L3 of the third sleeve member 26 is generally defined between the first end 60 and the second end 62 of the third sleeve member 26. The length L3 of the third sleeve member is preferably between 95 mm and 146 mm (3.75 inches and 5.75 inches); more preferably between 107 mm and 134 mm (4.25 inches and 5.25 inches). The outer perimeter P3 of the third sleeve member 26 defines the third access opening area; the third access opening area is generally between 368 and 1148 square mm (0.57 and 1.78 square inches).

Referring now to FIGS. 13-15, the fourth sleeve member or outer portal member 18 of the nested arrangement 12 is illustrated. The outer portal member 18 generally includes a sleeve portion 70 having a first end 82 and a second end 84. The sleeve portion 70 defines an elongated aperture 74 that extends from the first end 82 to the second end 84.

A handle portion 72 of the outer portal member 18 is located at the second end 84 of the sleeve portion 70. The handle portion 72 can include a plurality of holes 80. The holes 80 provide locations at which other surgical tools (not shown) can be attached for use during the surgical procedure.

In general, the outer portal member 18 has an overall width W4, an overall height H4, and an overall length L4, although the disclosed principles can be applied in a variety of sizes and applications. The width W4 of the outer portal member 18 is shown in FIG. 15, and is preferably between 30 mm and 68 mm (1.19 inches and 2.69 inches); more preferably between 49 mm and 56 mm (1.94 inches and 2.19 inches). The height H4 of the outer portal member 18 is also shown in FIG. 15, and is preferably between 20 mm and 25 mm (0.787 inches and 0.987 inches); more preferably between 20 mm and 22 mm (0.812 inches and 0.862 inches). The length L4 of the outer portal member 18 is generally defined between the first end 82 and the second end 84 of the outer portal member 18. The length L4 of the outer portal member is preferably between 97 mm and 149 mm (3.85 inches and 5.85 inches); more preferably between 110 mm and 136 mm (4.35 inches and 5.35 inches). The outer perimeter P4 of the outer portal member 18 defines the fourth or final access opening area; the fourth or final access opening area is generally between 477 and 1348 square mm (0.74 and 2.09 square inches).

In use, the surgical access instrument 10 provides access to first and second pedicle sites at a spinal column area or region. To begin a procedure, the first placement wire 14 is advanced through a patient's skin tissue and muscle until the wire 14 is positioned at a selected first pedicle site (e.g. B1 in FIG. 19) of a first vertebral body V1. The second placement wire 16 is positioned at a corresponding upper or lower second pedicle site (e.g. B2 in FIG. 19) of an adjacent vertebral body V2. The first and second pedicle sites are located a general distance D apart from one another. The site of the access opening is located at the region defined generally between and adjacent to the first and second placement wires 14, 16.

While first ends of the first and second placement wires 14, 16 are positioned at the first and second pedicle locations, opposite ends of the placement wires 14, 16 are inserted within the first and second apertures 34, 36 at the first end 28 of the blade member 20. The blade member 20 slides along the first and second placement wires 14, 16 in a first direction (represented by arrow A in FIG. 2) until the blade member 20 is adjacent to the skin tissue located between the first and second placement wires 14, 16. As the blade member 20 is further advanced toward the first and second pedicle sites, the blade edge 22 provides an initial incision through the skin tissue and muscle to the spinal column area. The surgeon can use hand force or a tapping hammer, for example, to advance the blade member along the placement wires 14, 16 to a desired depth.

When the blade member 20 is position at the desired depth adjacent to the spinal column area, the first end 50 of the second sleeve member 24 is positioned over the second end 30 of the blade member 20 (FIG. 2). The second sleeve member 24 slides along the blade member 20 in the first direction A until the second sleeve member 24 is adjacent to the initial incision in the skin tissue. As the second sleeve member 24 is further advanced toward the spinal column area, the tapered first end 50 of the second sleeve member 24 is introduced into the initial incision and begins to enlarge the incisional area. The incisional area is incrementally enlarged to the second opening area defined by the outer perimeter of the second sleeve member 24.

The second sleeve member 24 is inserted to a desired depth adjacent to the spinal column area, however cannot be inserted a depth exceeding the depth of the blade member 20. That is, the stop structures 46 of the second sleeve member 24 contact the shoulders 44 of the blade member 20 to limit the insertion depth of the second sleeve member.

When the second sleeve member 24 is position at the desired depth adjacent to the spinal column area, the first end 60 of the third sleeve member 26 is positioned over the second end 52 of the second sleeve member 24 (FIG. 2). The third sleeve member 26 slides along the second sleeve member 24 in the first direction A until the third sleeve member 26 is adjacent to the access opening in the skin tissue. As the third sleeve member 26 is further advanced toward the spinal column area, the tapered first end 60 of the third sleeve member 26 is introduced into the access opening and begins to enlarge the access opening. The access opening is incrementally enlarged from the second opening area to the third opening area defined by the outer perimeter of the third sleeve member 26.

The third sleeve member 26 is inserted to a desired depth adjacent to the spinal column area, however cannot be inserted a depth exceeding the depth of the second sleeve member 24. That is, the stop structures 66 of the third sleeve member 26 engage the notches 56 of the second sleeve member 24 to limit the insertion depth of the third sleeve member 26.

Similar to the preceding steps, when the third sleeve member 26 is position at the desired depth adjacent to the spinal column area, the first end 82 of the outer portal member 18 is positioned over the second end 62 of the third sleeve member 26 (FIG. 2). The outer portal member 18 slides along the third sleeve member 26 in the first direction A until the outer portal member 18 is adjacent to the access opening in the skin tissue. As the outer portal member 18 is further advanced toward the spinal column area, the first end 82 of the outer portal member 18 is introduced into access opening and begins to enlarge the access opening. The access opening is incrementally enlarged from the third opening area to the final opening area defined by the outer perimeter of the outer portal member 18.

When the portal member 18 has been positioned at the desired depth adjacent to the spinal column area, each of the members 18, 20, 24, and 26 are in the nested configuration, generally shown in FIG. 1. The access opening to the first and second pedicle sites at the spinal column area has been incrementally expanded to minimized incisional trauma and blood loss.

To continue the surgical procedure, each of the blade member 20, the second sleeve member 24, and the third sleeve member 26, is removed from the elongated aperture 74 of the portal member 18. Removing all three members 20, 24, and 26 can be accomplished by simply grasping the handle 32 of the blade member 20 and pulling the blade member 20 out from the aperture 74 of the outer portal member 18.

In particular, each of the blade, second sleeve and third sleeve members 20, 24, 26 are interconnected when moved in a second direction B (FIG. 1) relative to the outer portal member 18. That is, the shoulders 44 of the blade member 20 contact the pins 48 of the second sleeve member 24, and the notches 56 of the second sleeve member 24 engage the pins 68 of the third sleeve member 26 to form an interconnection that permits all three nested members 20, 24, 26 to be simultaneously removed from the aperture 74 of the outer portal member 18. Thus, as a surgeon pulls the blade member 20 from the aperture 74, the blade member 20 interconnects with the second sleeve member 24 and the second sleeve member interconnects with the third sleeve member 26 so that the three nested and interconnected members 20, 24, 26 can be removed at the same time.

When the three nested members 20, 24, and 26, are removed from the elongated aperture 74 of the outer portal member 18, the surgeon now has access to first and second pedicle sites at the spinal column area. The access is provided through the elongated aperture 74; thereby the elongated aperture 74 of the outer portal member 18 is sized and configured to correspond to the distance (D) between the first and second pedicle sites. More preferably, the elongated aperture 74 provides access to each of the first and second pedicle sites and the immediate surrounding area of each pedicle site at the spinal column area. In the illustrated embodiment, the elongated aperture 74 is sized and configured to receive and guide pedicle screws into the first and second vertebral bodies at the first and second pedicle sites.

It is to be understood that the placement wires 14, 16 may or may not be removed from the elongated aperture 74 with the three nested members 20, 24, 26. In some procedures, pedicle screws having a bore extending through the screw shaft are positioned on the placement wires. The placement wires therein act as guide wires to direct the pedicle screws to the first and second pedicle sites. In other procedures, the first and second placement wires 14, 16 are removed with the three nested members 20, 24, 26 and the screws are engaged by an appropriate driving tool and positioned down into the aperture to the first and second pedicle sites. In yet another alternative, the placement wires 14, 16 can be removed from the blade member 20 after the blade member 20 has been properly positioned adjacent to the spinal column area.

The pedicle screws can include a variety of pedicle screw configurations known in the art. Typically the diameter of pedicle screws range between about 5 mm and 8 mm. These specific dimensions are merely illustrative of normal configurations and can be varied as needed. Accordingly, the elongated aperture 74 of the outer portal member 18 can be varied to accommodate the variety of pedicle screw configurations.

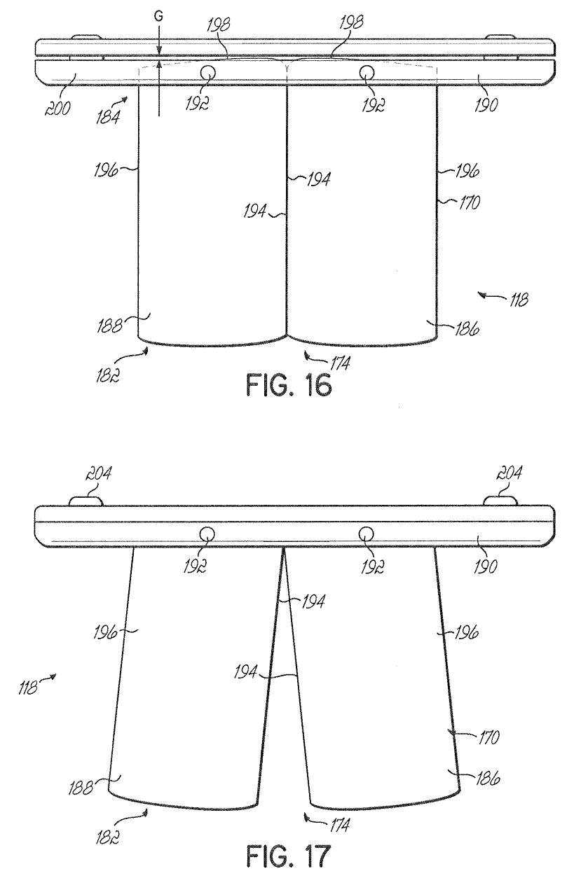

Referring now to FIGS. 16-18, a second embodiment of an outer portal member or fourth sleeve member 118 is illustrated. In this embodiment, the outer portal member 118 generally includes a sleeve portion 170 having a first end 182 and a second end 184. The sleeve portion 170 defines an elongated aperture 174 that extends from the first end 182 to the second end 184. The second outer portal member embodiment 118 generally has similar overall width, height, and length dimensions as the first outer portal member 18 shown in FIGS. 13-15.

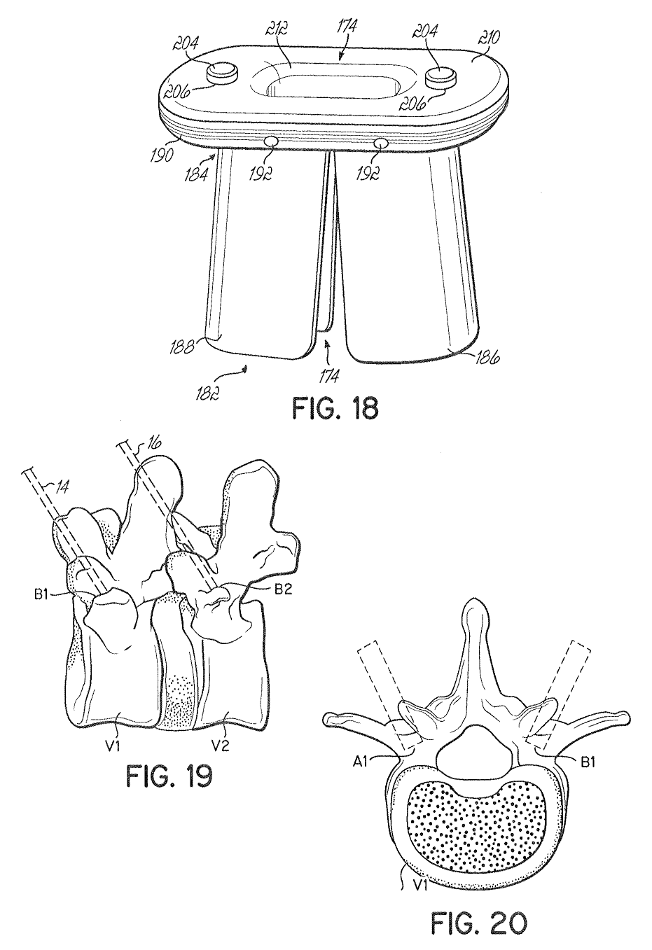

The sleeve portion 170 illustrated in the second embodiment, however, includes a first sleeve section 186 and a second sleeve section 188 that define the elongated aperture 174. The first and second sleeve sections 186,188 are coupled to a flange or collar 190 at pivot locations 192. Each of the first and second sleeve sections 186,188 is configured to rotate or pivot, relative to the collar 190, from a retracted position (shown in FIG. 16) to a distended position (shown in FIGS. 17 and 18).

The second end 184 of each of the sleeve sections 186, 188 is angled such that an inner region 194 of each section is longer than an outer region 196. In other words, the second end 184 of each section has an oblique edge construction 198 (partially shown in FIG. 16) relative to the inner and outer regions 194, 196 of the first and second sleeve sections 186, 188.

The outer portal member 118 further includes a clamp plate 210 positioned adjacent to the collar 190. Typically, the clamp plate 210 is positioned in relation to the collar 190 so that a gap G is provided between the collar 190 and the clamp plate 210. Alignment spacers 202 in cooperation with holes 206 formed in the clamp plate 210 properly orient the clamp plate 210 relative to the collar 190 so that an opening 212 in the clamp plate 210 is aligned with the elongated aperture 174 of the sleeve portion 170. The alignment spacers 202 can also be configured to maintain the gap G between the collar 190 and the clamp plate 210. For example, the alignment spacers 202 can be configured to provide a sufficient interference fit with the holes 206 formed in the clamp plate 210 such that the clamp plate 210 seats in an offset position from the collar 190 when no force is applied. In the illustrated embodiment, the spacers 202 are pegs 204 extending from a first surface 200 of the collar 190.

As shown in FIG. 16, when the gap G is provided between the collar 190 and the clamp plate 210, the first and second sleeve sections 186, 188 remain in the retracted position. In the retracted position, the outer portal member 118 can be introduced into an access opening area as previously described with respect to the first outer portal member embodiment.

When the outer portal member 118 is positioned adjacent to the spinal column area at the desired depth, and the three nested members 20, 24, 26 are removed from the elongated aperture 174, the first and second sleeve sections 186, 188 can be outwardly distended to further expose the first and second pedicle sites. In particular, the clamp plate 210 can be forcibly positioned to contact the first surface 200 of the collar 190 (FIGS. 17 and 18). As the clamp plate 210 is forced towards the collar 190, the clamp plate 210 contacts the oblique edge construction 198 of the second end 184 of the first and second sleeve sections 186, 188. The force from the clamp plate 210 pivots the first end 182 of the first and second sleeve members 186, 188 outward away from one another. That is, the second end 184 of the first and second sleeve members 186, 188 pivot about pivot locations 192, and the first end 182 of the first and second sleeve members 186, 188 rotate in opposite directions from one another.

The clamp plate 210, spacers 198, and collar 190 can be configured such that a surgeon can forcibly position the outer portal member 118 in the distended position by hand, or such that a clamp (not shown) is required to press the clamp plate 210 toward the collar 190. The pivoting design of this second outer portal member embodiment provides a greater access opening adjacent to the spinal column area without having to expand the access opening in the tissue and muscle region of the patient's back. This is advantageous in further reducing trauma in situations where access to a larger spinal column area is needed.

The above specification provides a complete description of SPINAL ACCESS INSTRUMENT. Since many embodiments of the invention can be made without departing from the spirit and scope of the invention, the invention resides in the claims hereinafter appended.

Various surgical procedures may be performed on the surgical site through the outer portal member 18. For example, spinal stabilization devices may be installed onto the spine through the outer portal member 18 to stabilize a motion segment of the spine. A dynamic stabilization is a device that acts to stabilize the spine while permitting movement and flexibility of the spine. Such device may also be known as non-fusion devices. In one embodiment, a dynamic stabilization device such as the Dyneses® Dynamic Stabilization System may be installed through the portal (available from Zimmer, Inc. of Warsaw, Ind.).

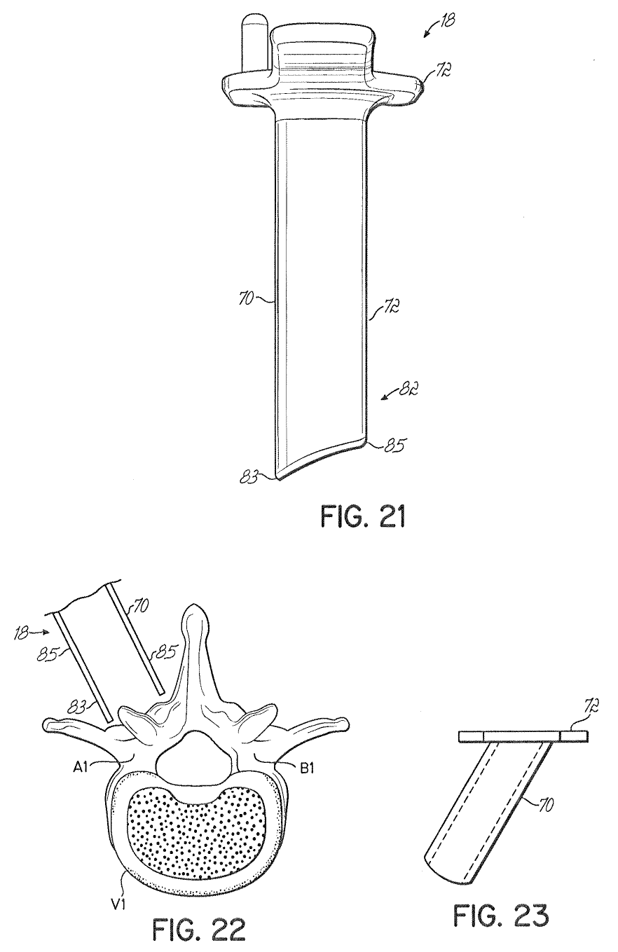

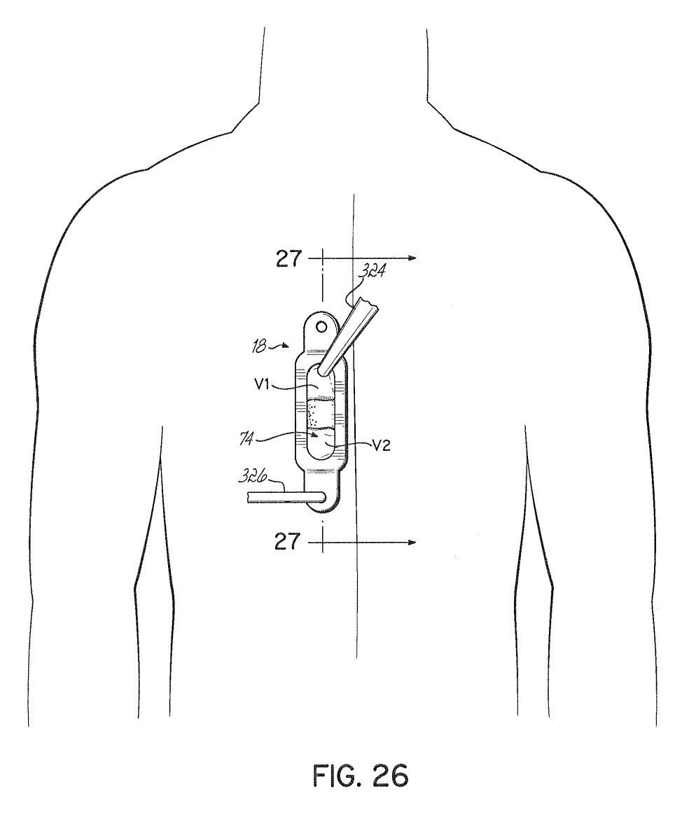

In one embodiment, the outer portal member 18 and in particular the length and width of the aperture 74 is sized and shaped to facilitate installing a dynamic stabilization device such as the dynamic stabilization device shown in FIG. 26 onto the spine. FIGS. 21-25 show examples of outer portal members 18 which are so sized and shaped and which may include additional features to facilitate installation of a dynamic stabilization device.

FIG. 21 shows an outer portal member 18 according to another embodiment of the present invention. The outer portal member 18 is similar to those shown in the preceding figures and like parts are given like numbering. However, the first end 82 of the outer portal member 18 is asymmetric, such that a first side 83 is longer than a second side 85. The asymmetric first end 82 may be employed to contour around the bony anatomy of the pedicles, as shown in FIG. 22. The asymmetric first end 82 may also be employed to accommodate an angle of insertion of the outer portal member 18 that is angled relative to the spine, as shown in FIG. 22. The second side 83 of the outer portal member 18 is shown in FIG. 21, and is preferably between 5 and 15 mm longer than the first side 85; more preferably between 8 and 12 mm longer than the first side 85.

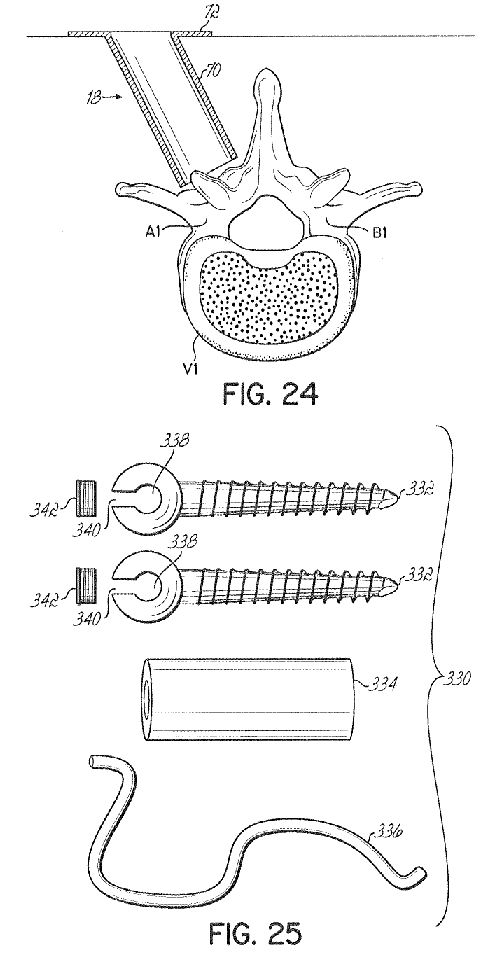

FIG. 23 shows an outer portal member 18 according to another embodiment of the present invention. The outer portal member 18 is similar to those shown in the preceding figures, and like parts are given like numbering. However, the handle portion 72 of outer portal member 18 is angled relative to the sleeve portion 70. As shown in FIG. 23, an outer portal member 18 having this configuration accommodates an angle of insertion that is angled relative to the spine. While the sleeve portion 70 is inserted into the patient at an angle relative to the spine that is non-perpendicular, as shown in FIG. 24, the handle portion 72 rests flush against the patient's skin to provide increased stability. The angle of the handle portion 72 relative to the sleeve portion 70 is shown in FIG. 23, and is preferably between 30° and 60°; more preferably between 40° and 50°.

The spinal access instrument 10 may be made of a variety of sterilizable materials, including, but not limited to, stainless steel, plastic and titanium. The outer portal member 18 is preferably made of a material that is radiolucent, such as titanium. A radiolucent material such as titanium is translucent when imaged with fluoroscopy. This construction permits the use of fluoroscopy to image or visualize the surgical site during installation of a spinal device without the outer portal member 18 blocking the view.

FIG. 25 illustrates the components of an exemplary dynamic stabilization system 330 which may be installed through an outer portal member 18 according to various embodiments of the present invention. Dynamic stabilization system 330 includes pedicle screws 332, a spacer 334 and a cord 336. The pedicle screws 332 are bone-engaging members and are attached to the vertebrae to anchor the spacer 334 and cord 336. The pedicle screws 332 include a through-hole 338 for receiving the cord 336 and an opening 340 to the through-hole 338 for receiving a set screw 342. The set screw 342 is threaded into the opening 340 to fix the cord 336 to the pedicle screw 332. The spacer 334 is used to hold the segment in a more natural anatomical position and to control the spine in extension. The cord 336 controls forward flexion movement.

FIGS. 26-31 show various stages of a surgical procedure in which the dynamic stabilization system 330 is installed or implanted onto the spine through the outer portal member 18. As shown in FIG. 26, the patient is placed into a prone position. Other positions, such as a knee-chest position, are also acceptable provided that care is taken to preserve the natural lordosis in the lumbar spine as well as to avoid any pressure on the abdominal cavity that might results in excessive bleeding. The illustrative example shows the incision at a lumbar region of the spine; however, it is contemplated that the methods and apparatuses described herein are equally adapted to use in other regions of the spine.

The outer portal member 18 is installed to provide access to the pedicles of adjacent vertebrae V1 and V2 as previously described.

It should be realized that in some embodiments, however, the spinal access instrument 10 is installed without the use of first and second placement wires 14, 16. For example, the blade member 20 may be held in place manually and advanced toward the first and second pedicle sites. Alternately, a scalpel or other cutting device may be employed to create an incision extending between the first and second pedicle sites. The blade member 20 may then be advanced into the incision to access the first and second pedicle sites and to stretch the incision. The second, third and fourth sleeve members 24, 26 and 18 are slid over the blade member 20 as previously described.

Optionally, after making an initial incision with a scalpel or other cutting device, the surgeon may use their fingers to push aside muscle tissue above the vertebrae rather than cutting through the muscle. Once the appropriate muscle tissue has been pushed aside, the blade member 20 is advanced into the incision. The blade member 20 holds the displaced tissue away from the surgical site while the second, third and fourth sleeve members 24, 26 and 18 are advanced into the incision. This method may reduce trauma to the patient.

The spinal access instrument 10 may be initially inserted at an angle relative to the spine so that a long axis of the outer portal sleeve portion 70 is oriented at an angle to the spine as shown in FIG. 24. In one embodiment, the outer portal sleeve portion 70 is oriented at an angle of between 30° and 60° relative to the spine. In this manner, the pedicle screws 332 are more easily driven into the pedicles at a corresponding angle. This provides increased strength and stability of the connection between the bones of the vertebrae and the pedicle screws 332.

As shown in FIG. 26, a lighting device 324 such as a fiber optic light source may be attached to the outer portal member 18 so as to illuminate the exposed surgical site within the elongated aperture 74. Additional accessories may be attached to the outer portal member 18, including, for example, a fluid removal or suction device or an endoscopic viewing device. A stabilization arm 326 may be attached to the outer portal member 18 and attached to the patient's body, the surgical table or to some other stationary device to stabilize the position of the outer portal member 18 and prevent dislodgement of the outer portal member 18 from the patient.

A tool may be employed to remove or push aside tissue obscuring the spine so as to expose portions of the spine within the elongated aperture 74. For example, a scraper, rongeur or electrocautery device may be employed to expose the facets, pedicles or other appropriate portions of the vertebrae so as to permit installation of the spinal stabilization device. Such a tool is maneuvered by the physician through the outer portal member 18 to access the surgical site. Furthermore, such tools may include a lateral offset such that the tool does not obstruct the view of the surgical site through the outer portal member 18.

A spacer template or guide 344 may be employed to determine the correct position of the pedicle screws 332 relative to the facet joints. For example, the template 344 may be configured to facilitate driving the pedicle screws 332 into the correction portion of the vertebrae and at a chosen angle, as previously described. With the template 344 in position, a tool such as a bone awl may be employed to pierce the cortical bone of the exposed pedicles. A probe may then be employed to establish a channel for insertion of each of the pedicle screws 332 into the pedicles. The orientation of the probe generally determines the ensuing orientation of the pedicle screws 332. Fluoroscopy or X-ray visualization devices may be employed to determine the position of the vertebrae relative to the outer portal member 18 and to facilitate proper placement of the bone awl and probe relative to the vertebrae. Doing so increases the likelihood that the pedicle screws 332 will be subsequently installed in the correct orientation and position.

The length of the pedicle screws 332 depends on the patient morphology, and may be from about 35 to about 55 mm. Care should be taken so as to avoid inserting the probe deeper into the pedicles than the length of the intended pedicle screw 332. To avoid over-insertion of the probe, the probe may be provided with depth markings corresponding to pedicle screw lengths. Alternately, a sleeve provided with depth markings at a proximal end may be fit over the probe.

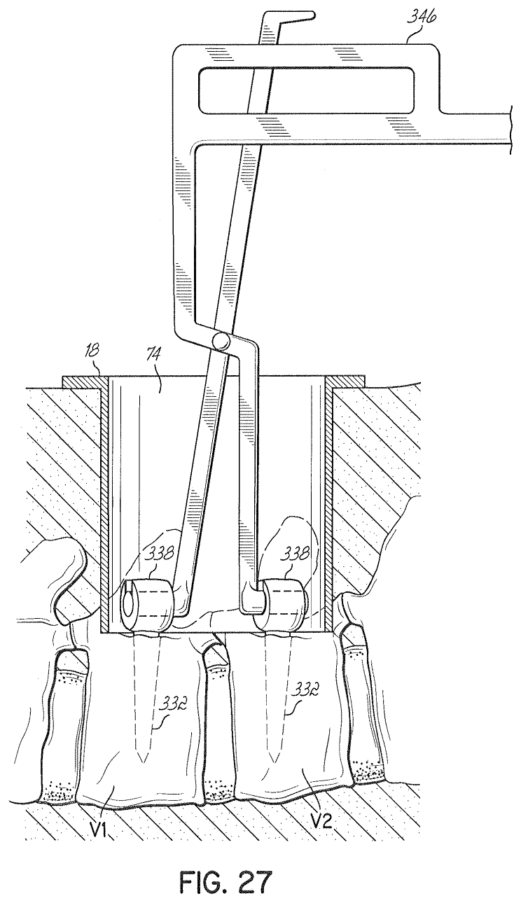

The probe is removed and the intactness of the pedicle wall may be checked with a pedicle sound. If the pedicle wall is determined to be sufficiently intact, a first pedicle screw 332 is driven into the pedicle, as shown in FIG. 27. Optionally, a guide device such as a guide pin is coupled to the pedicle screw 332 (not shown). The guide device may improve the orientation possibilities and may make subsequent instrument positioning easier. However, care should be taken to avoid over-tightening the guide device onto the pedicle screw 332, which could cause difficulty in loosening later.

A driving tool such as a screw driver is employed to drive the pedicle screws into the channel. A portion of the tool may be laterally offset to avoid blocking the user's view of the surgical site through the outer portal member 18 while the pedicle screws are driven into the pedicles. Optionally, a stabilizing device such as a T-handle is used in conjunction with the driving tool to facilitate insertion of the screw. Such a stabilizing device may reduce wobbling of the screw during tightening.

As shown in FIG. 27, in general, the pedicle screw 332 is advanced as deep as possible, i.e., when a head portion of the screw 332 is in contact with the bone. Care should be taken to avoid over-torquing the screws 332 or exerting an excessive bending load, as this could fracture the pedicle. After the pedicle screw 332 is driven fully into the channel, the guide pin or other guiding or driving device may be removed. Alternately, the guide device may remain coupled to the pedicle screw 332 until the remainder of the stabilization device is assembled.

The process described above is repeated to install the second pedicle screw 332 into place. The pedicle screws 332 should be aligned with one another so that the through-holes 338 are aligned and will allow passage of the cord 336 therethrough.

After insertion of the pedicle screws 332, the distance between the pedicle screws 332 is measured to determine the appropriate length of the spacer 334. A drag indicator 346 as shown in FIG. 23 may be employed to measure the distance between the screws 332 and to assess the movement in the facets in distraction and compression. As shown in FIG. 27, the ends of the drag indicator 346 are inserted into the through-holes 338 of the pedicle screws 332. The distance between the screws 332 is measured under a slight distraction force. The size of the spacer 334 is chosen relative to the distance between the pedicle screws 332 as well as to achieve various physiological effects. For example, the spacer 334 might be slightly oversized to distract the segments to create parallel vertebral end plates. Alternately, the spacer 334 might slightly oversized to distract the segments to create a neutral facet joint position. An appropriately-sized pre-cut spacer 334 may be chosen, or the spacer 334 may be custom cut according to the patient's measurements.

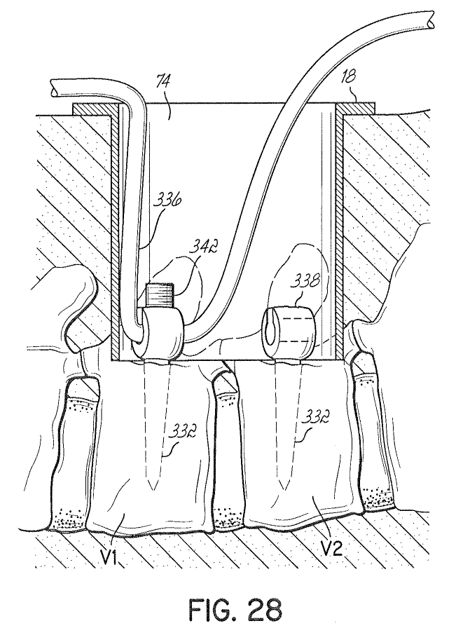

As shown in FIG. 28, the flexible cord 336 is threaded through the through-hole 338 of a first of the pedicle screws 332. A cord threader may be used to guide the cord 336 into the through-hole 338. Because the aperture 74 is oblong and aligned in with the an axis extending between the pedicle screws 332, the cord 336 remains aligned to the pedicle screws 332 and organized during the procedure to reduce kinking and misalignment of the cord 336.

A set screw 342 is tightened into the pedicle screw opening 340 to fix the cord 336 to the pedicle screw 332. An anti-torque device may be inserted over the guide pin (if in place) and screw 332 to overcome any binding of the guide pin. The set screw 342 may be tightened with a laterally offset driver or other tool so as to avoid obscuring user's view of the exposed incision through the outer portal member 18.

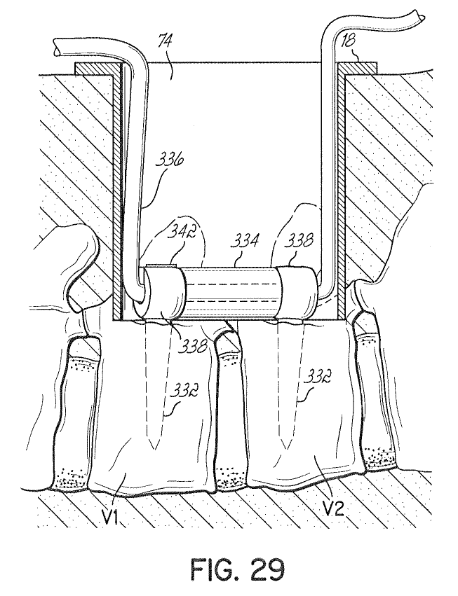

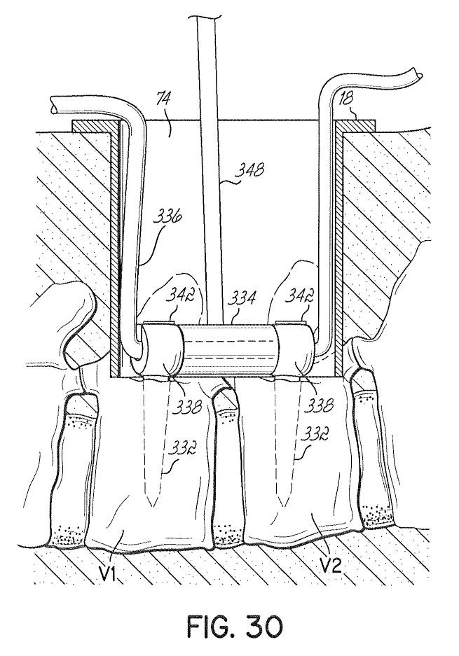

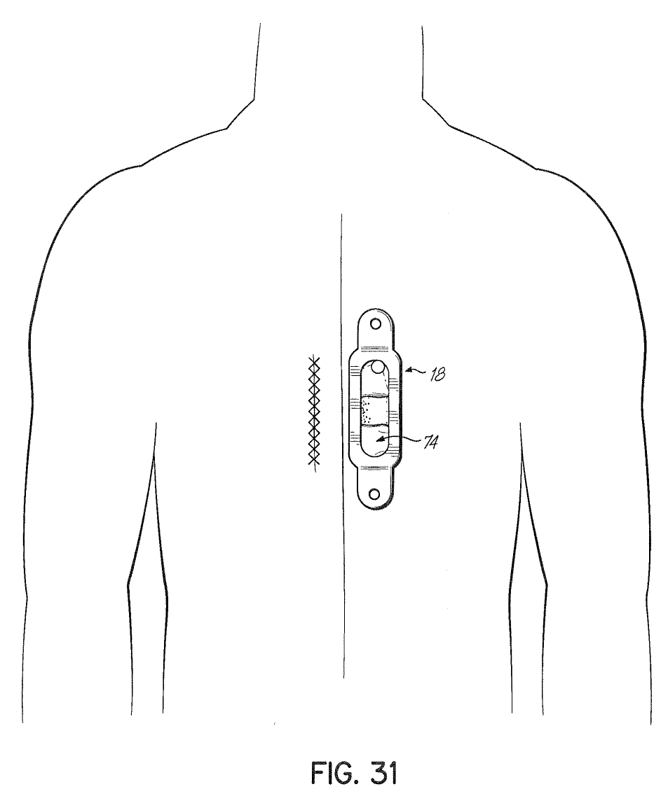

As shown in FIG. 29, the spacer 334 is then threaded onto the cord 336 and advanced over the cord 336 into the portal opening 14. The spacer 334 is advanced into a position against the first screw 332. As shown in FIG. 30, the cord 336 is then threaded through the through-hole 338 of the second pedicle screw 332. The cord 336 is tensioned until the spacer 334 is positioned between the first and second pedicle screws 332. A cord tensioner may be employed to tension the cord 336 and to pull the spacer 334 into position between the pedicle screws 332. Other tools such as a depressor and/or forceps 348 may be employed to grip the spacer 334 and position it between the pedicle screws 332. Again, such tools may be laterally offset to avoid blocking the user's view of the incision through the outer portal member 18. In other embodiments, the spacer 334 may be threaded onto the cord 336 prior to the first set screw 342 being tightened onto the opening 340 of the first pedicle screw 332.

Tension is maintained on the cord 336 and may be adjusted according to the desired patient result. A second set screw 342 is inserted into the opening 340 of the second pedicle screw 332 and tightened to fix the spacer 334 into position between the pedicle screws 332.

In other embodiments, the cord 336 is threaded through second pedicle screw 332 before the spacer 334 is positioned between the pedicle screws. This avoids blocking the through-hole 338 of the second pedicle screw 332 while attempting to thread the cord 336. After the cord 336 is threaded through the second pedicle screw 332, the spacer 334 is maneuvered into position, the cord 336 is tensioned and the second set screw 342 is tightened. The cord 336 may be further tensioned and either or both of the set screws 342 tightened further.

Excess portions of the cord 336 may be trimmed and removed. The outer portal member 18 may then be removed and the incision closed.

As shown in FIG. 31, the procedures previously described herein may be repeated on the adjacent pedicles of the vertebrae to stabilize the adjacent segment. Thus, a second incision is made approximately 3.5 cm lateral to the spinous process on the other side of the spine. The incision is dilated as previously described, the outer portal member 18 is inserted and a second spinal stabilization device 330 is installed on the pedicles of the vertebrae.

The procedures previously described herein may be employed to install a multi-level spinal stabilization device. A multi-level device is one in which multiple vertebral motion segments along the longitudinal axis of the spine are stabilized. Thus, the procedures previously described herein may be employed to install spinal stabilization devices on segments above and/or below the stabilized motion segment to stabilize greater portions of the spine.

The present method is not limited to installation of a dynamic stabilization device as described and shown in FIG. 21. Other types of dynamic spinal stabilization devices that permit motion and flexion of the spine may be installed on the spine according to the methods and devices previously described.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method of stabilizing a motion segment of the spine, the method comprising:
forming a posterior incision lateral to a spinous process of the spine;
stretching a length and width of the incision;
inserting a portal into the incision, the portal having an open inner area sized and shaped to provide access to the pedicles of adjacent vertebrae and having a long axis aligned with an axis extending between the pedicles; and
implanting a dynamic spinal stabilization device onto the pedicles through the open inner area of the portal.

2. The method of claim 1 further comprising repeating the method on an adjacent motion segment of the spine.

3. The method of claim 1 wherein forming a posterior incision further comprising manually displacing muscle tissue overlaying the spinous process of the spine.

4. The method of claim 1 wherein stretching the incision includes inserting incrementally larger sleeve members into the incision.

5. The method of claim 1 wherein inserting the portal further comprises inserting the portal at an angle of from about 30° to about 60° relative to the spine.

6. The method of claim 1 wherein the spinal stabilization device comprises:
a pair of bone-engaging members;
a flexible cord securable to the bone-engaging members; and
a spacer threaded onto the cord between the bone-engaging members.

7. The method of claim 6 wherein implanting the spinal stabilization device further comprises:
implanting the bone-engaging members to adjacent pedicles with a gap there-between;
attaching the cord to a first of the bone-engaging members;
advancing the spacer over the cord into the gap between the bone-engaging members;
tensioning the cord; and
attaching the cord to the other of the bone-engaging members.

8. The method of claim 1 wherein implanting the bone-engaging members further comprises imaging the position of the bone-engaging members relative to the pedicles through the outer portal member.

9. The method of claim 1 wherein implanting the spinal stabilization device further comprises using laterally offset implantation tools.

10. A method of stabilizing a motion segment of the spine, the method comprising:
forming a posterior incision lateral to a spinous process of the spine;
inserting incrementally larger sleeve members into the incision to stretch a length and width of the incision;
inserting a portal into the incision, the portal having an open inner area sized and shaped to provide access to the pedicles of adjacent vertebrae and having a long axis aligned with an axis extending between the pedicles; and
implanting a dynamic spinal stabilization device onto the pedicles through the open inner area of the portal.

11. The method of claim 10 further comprising repeating the method on an adjacent motion segment.

12. The method of claim 10 wherein the dynamic stabilization device comprises:
a pair of bone-engaging members;
a flexible cord securable to the bone-engaging members; and
a spacer threaded onto the cord between the bone-engaging members.

13. The method of claim 12 wherein implanting the spinal stabilization device further comprises:
attaching the bone-engaging members to adjacent pedicles with a gap therebetween;
attaching the cord to a first of the bone-engaging members;
advancing the spacer over the cord into the gap between the bone-engaging members;
tensioning the cord; and
attaching the cord to the other of the bone-engaging members.

14. The method of claim 13 further comprising:
measuring the gap between the first and the second bone-engaging members; and
selecting a spacer sized appropriately relative to the gap.

15. A method of stabilizing a motion segment of the spine, the method comprising:
forming a posterior incision lateral to a spinous process of the spine;
inserting a portal into the incision, the portal having a sleeve portion defining an open inner area;
implanting a dynamic stabilization device onto the spine through the open inner area of the portal, including:
attaching a pair of bone-engaging members to adjacent pedicles with a gap therebetween;
attaching a flexible cord to a first of the bone-engaging members;
advancing a spacer over the cord into the gap between the bone-engaging members;
tensioning the cord; and
attaching the cord to the other of the bone-engaging members.

16. The method of claim 15 further comprising stretching a length and width of the incision.

17. The method of claim 15 further comprising inserting the sleeve portion of the portal into the incision at an angle to an axis of the spine.

18. The method of claim 15 further comprising repeating the method on an adjacent motion segment of the spine.

19. The method of claim 15 further comprising:
measuring the gap between the bone-engaging members; and
selecting a spacer sized appropriately relative to the gap.

20. The method of claim 15 wherein implanting the spinal stabilization device further comprises imaging the position of the bone-engaging members relative to the pedicles.
